(19) United States
(12) Patent Application Publication
(10) Pub. No.: US 2026/0015580 A1
(43) Pub. Date: Jan. 15, 2026

(54) Methods of Producing and Using Avian Embryonic Stem Cells and Avian Telencephalic Organoids

(71) Applicant: The Regents of the University of California, Oakland, CA (US)

(72) Inventors: Mohammed Mostajo-Radji, Santa Cruz, CA (US); Hunter Schweiger, Santa Cruz, CA (US)

(21) Appl. No.: 18/997,725

(22) PCT Filed: Jul. 25, 2023
(86) PCT No.: PCT/US2023/070947

§ 371 Date: Jan. 22, 2025

Related U.S. Application Data
(60) Provisional application No. 63/392,046, filed on Jul. 25, 2022.

(51) Int. Cl. C12N 5/0735; C12N 5/00; G01N 33/50

(52) U.S. Cl. CPC C12N 5/0606; C12N 5/0018; G01N 33/5073; G01N 33/5088; C12N 2500/84; C12N 2501/115; C12N 2501/15; C12N 2501/727; C12N 2506/02; C12N 2510/00; C12N 2539/00

(57) ABSTRACT

Provided are methods of producing avian embryonic stem cells. In certain embodiments, the methods comprise culturing epiblast cells obtained from a stage IX-XI avian embryo in a cell culture medium present in a feeder-free cell culture container. Also provided are methods of producing two-dimension cultures, spheroids, or organoids from the avian embryonic stem cells produced according to the methods. For example, provided are methods of producing avian telencephalic organoids from the avian embryonic stem cells. Also provided are methods of using the avian embryonic stem cells, spheroids and organoids produced according to the methods of the present disclosure. For example, provided are screening methods comprising contacting the avian embryonic stem cells, spheroids or organoids with a test agent, and assessing for an effect of the test agent on the avian embryonic stem cells, spheroids or organoids.

### CROSS-REFERENCE TO RELATED APPLICATIONS

This application claims the benefit of U.S. Provisional Patent Application No. 63/392,046, filed Jul. 25, 2022, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, UCSC-403WO_SEQ_LIST, created on Jul. 25, 2023 and having a size of 4,292 bytes. The contents of the Sequence Listing XML are incorporated herein by reference in their entirety.

### SUMMARY

Provided are methods of producing avian embryonic stem cells. In certain embodiments, the methods comprise culturing epiblast cells obtained from a stage IX-XI avian embryo in a cell culture medium present in a feeder-free cell culture container. Also provided are methods of producing two-dimension cultures, spheroids, or organoids from the avian embryonic stem cells produced according to the methods. For example, provided are methods of producing avian telencephalic organoids from the avian embryonic stem cells. Also provided are methods of using the avian embryonic stem cells, spheroids and organoids produced according to the methods of the present disclosure. For example, provided are screening methods comprising contacting the avian embryonic stem cells, spheroids or organoids with a test agent, and assessing for an effect of the test agent on the avian embryonic stem cells, spheroids or organoids.

### BRIEF DESCRIPTION OF THE FIGURES

FIG. 1: Schematic overview of a method of producing avian embryonic stem cells according to some embodiments of the present disclosure.

FIG. 2: Image of a stage IX-XI developing avian embryo from which avian embryonic stem cells may be derived.

FIG. 3: Images of colonies of chicken pluripotent embryonic stem cells produced according to embodiments of the methods of the present disclosure. In this example, the cells were cultured and passaged 9 times for over the course of 1 month.

FIG. 4: Images of alkaline phosphatase-stained HE2M38 chicken pluripotent embryonic stem cells produced according to embodiments of the methods of the present disclosure. Cells were cultured and passaged 8-9 times over the course of 1 month.

FIG. 5: Image of telencephalic chicken organoids generated from passage 6 HE2M38 cells. Brightfield images were taken at differentiation day 10.

FIG. 6: Images of immunohistochemical staining for the neuronal markers Tubb3 and Mef2c in chicken telencephalic organoids at differentiation day 17.

FIG. 7: Images of immunohistochemical staining for the transcription factor Mef2c in chicken telencephalic organoids at differentiation day 17. Mef2c labels maturing neurons.

FIG. 8: Images of immunohistochemical staining for the transcription factor SATB2 and phosphorylated ribosomal protein S6 (pS6) in chicken telencephalic organoids at differentiation day 17.

FIG. 9: Images of immunohistochemical staining for the phosphorylated ribosomal protein S6 (pS6) in chicken telencephalic organoids at differentiation day 17. S6 phosphorylation is a downstream target of the mTOR pathway which is key for telencephalic neurogenesis.

FIG. 10: Images of immunohistochemical staining for the transcription factor SATB2 in chicken telencephalic organoids at differentiation day 17. SATB2 labels a subset of excitatory neurons in the dorsal pallium of the telencephalon.

FIG. 11: Images of immunohistochemical staining for glial fibrillary acidic protein (GFAP) in chicken telencephalic organoids at differentiation day 17. GFAP labels astroglial cells in the brain.

FIG. 12: Images of immunohistochemical staining for neuronal-specific microtubule protein beta tubulin class III (Tubb3) in chicken telencephalic organoids at differentiation day 17. Tubb3 labels axons.

FIG. 13: Formulation of the B27, GS21, and N2 supplements. Adapted from Sünwoldt et al. (2017) Front Mol Neurosci. 10:305.

FIG. 14: A) Amino acid alignment between human leukemia inhibitory factor (hLIF—top strand—SEQ ID NO: 1) and chicken leukemia inhibitory factor (cLIF—bottom strand—SEQ ID NO: 3) shows only a 43% conservation in protein sequence. B) Amino acid alignment between mouse leukemia inhibitory factor (mLIF—top strand—SEQ ID NO: 2) and chicken leukemia inhibitory factor (cLIF—bottom strand—SEQ ID NO: 3) shows only a 42% conservation in protein sequence.

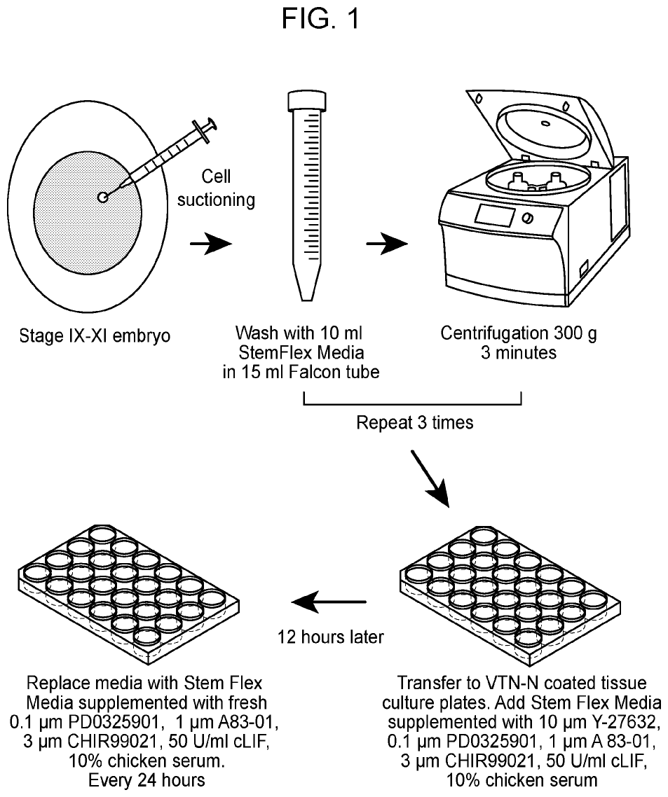

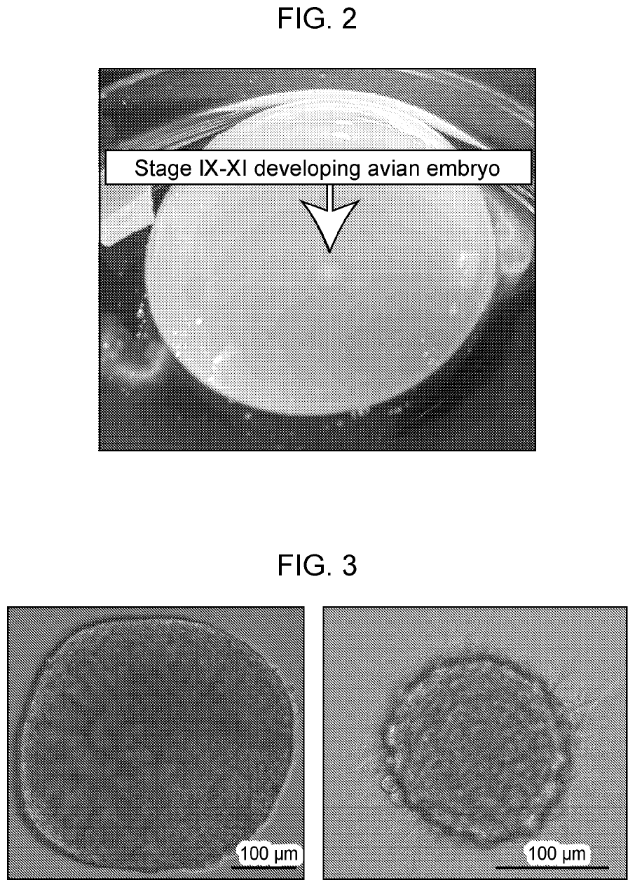

### DETAILED DESCRIPTION

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### METHODS OF PRODUCING AVIAN EMBRYONIC STEM CELLS

The present disclosure provides methods of producing avian embryonic stem cells. In some embodiments, the methods comprise culturing epiblast cells obtained from a stage IX-XI avian embryo in a cell culture medium present in a feeder-free cell culture container. A significant challenge to the production of avian embryonic stem cells is that they are traditionally cultured on a layer of feeder cells to prevent differentiation and to promote cell survival and proliferation.

Without feeder cells in the culture environment, stem cells will either die or differentiate into a heterogeneous population of committed cells. However, using feeder cells increases production costs, impairs scale-up, and produces mixed cell populations that require the stem cells to be separated from feeder cell components. The present disclosure is based in part on the development of methods of producing pluripotent avian embryonic stem cell lines in the absence of interfering feeder cells. Such pluripotent avian embryonic stem cell lines find a variety of uses, including but not limited to the differentiation of such cells into cells of a tissue/organ of interest, the production of spheroids, organoids and the like, all of which in turn find use as previously unavailable research and high-throughput screening tools. Details regarding the methods of the present disclosure will now be described.

In certain embodiments, the methods of the present disclosure comprise culturing epiblast cells obtained from a stage IX-XI avian embryo (e.g., a stage X avian embryo) in a cell culture medium present in a feeder-free cell culture container. According to some embodiments, the avian embryo is a chicken (Gallus gallus) embryo. As used herein, “epiblast cells” are cells obtained from the epiblast of an embryo. The epiblast is a tissue type derived from the blastodisc in birds. The avian embryo comes entirely from the epiblast. All three germ layers of the embryo proper are formed from the epiblast cells.

According to some embodiments, the epiblast cells were obtained from a stage IX-XI avian embryo. For example, the epiblast cells may have been obtained within 2 hours following ovopositioning, which represents stage IX-XI of avian development. As used herein, a “stage IX-XI” avian embryo or “stage X” avian embryo is based on the staging scale described in Eyal-Giladi & Kochav (1976) Dev Biol. 49(2):321-37, the disclosure of which is incorporated herein by reference in its entirety for all purposes. In certain embodiments, the methods comprise obtaining the epiblast cells. Approaches for obtaining such cells include, but are not limited to, breaking an avian egg within two hours of ovopositioning, manipulating the orientation of the yolk so that the blastocyst is face up, puncturing the fertilized egg through the area pellucida with a syringe, and removing the epiblast or cells thereof via suction with the syringe. The epiblast cells may be washed one or more times (e.g., via centrifugation) prior to culturing the cells. An example approach for obtaining epiblast cells from a stage IX-XI avian embryo is described in detail in the Experimental section below.

In certain embodiments, the cell culture medium comprises active FGF-2. Fibroblast growth factor 2 (FGF-2 or basic FGF) is a critical medium component for maintenance of a number of stem cell types, but is highly labile at 37° C. and prone to denaturation. According to some embodiments, the cell culture medium comprises active FGF-2, where the FGF-2 is active by virtue of being stabilized. Approaches for stabilizing FGF-2 are known and include, e.g., stabilization via encapsulation in microspheres (e.g., polyesters of glycolic and lactic acids (PLGA) microspheres), the use of supplements bearing sulfate/sulfonate groups, the use of heparin, and the like.

According to some embodiments, the cell culture medium comprises a cell-permeable MEK/ERK pathway inhibitor. Mitogen-activated protein kinase (MAPK) pathways are signaling cascades that regulate a wide variety of cellular processes, including proliferation, differentiation, apoptosis and stress responses. The MAPK pathway includes three main kinases, MAPK kinase kinase, MAPK kinase and MAPK, which activate and phosphorylate downstream proteins. MEK is a MAPKK that activates a MAPK (ERK), the final kinase in the RAS-RAF-MEK-ERK signaling pathway. In certain embodiments, the cell-permeable MEK/ERK pathway inhibitor is PD0325901. PD0325901 is a selective, cell permeable inhibitor of the MEK/ERK pathway that inhibits the activation and downstream signaling of MEK. See Barrett et al. (2008) Bioorg Med Chem Lett. 18(24):6501-4. It is a highly potent inhibitor, suppressing the phosphorylation of ERK in C26 cells at very low concentrations (IC₅₀=0.33 nM). According to some embodiments, the cell-permeable MEK/ERK pathway inhibitor is present in the cell culture medium in an amount of from 0.01 to 1 μM, e.g., from 0.05 to 0.5 μM, e.g., about 0.1 μM.

In certain embodiments, the cell culture medium comprises a TGF-β type I receptor signaling inhibitor, e.g., a cell-permeable TGF-beta type I receptor signaling inhibitor. TGF-β signaling is initiated by the binding of TGF-β to its serine and threonine kinase receptors, the type II (TβRII) and type I (TβRI) receptors on the cell membrane. Ligand binding leads to formation of the receptor heterocomplex. in which TβRII phosphorylates threonine and serine residues in the TTSGSGSG motif of TβRI and thus activates TβRI. The activated TβRI recruits and phosphorylates the R-Smad proteins, Smad2/3 for TGF-β and activin signaling while Smad1/5/8 for BMP signaling, which then form a heterocomplex with the Co-Smad Smad4. The Smad complexes are then translocated into the nucleus to regulate transcription of the target genes in cooperation with other co-factors. For each member of the TGF-β family, they have their own type I and type II receptors. Among the seven type I receptors, which are also referred to as activin receptor-like kinases (ALKs), TβRI/ALK5 can mediate TGF-β signaling with the TGF-β type II receptor TβRII to activate Smad2/3 in universal cell types, while in endothelial cells ALK1 functions with TβRII to activate Smad1/5/8 for TGF-β signaling. In response to BMPs, ALK2/3/6 can activate Smad1/5/8 with the type II receptors BMPRII, ActRII and ActRIIB. ALK4/7 can activate Smad2/3 with ActRII and ActRIIB to mediate activin/Nodal signaling.

According to some embodiments, the TGF-type I receptor signaling inhibitor is an ALK inhibitor. For example, in certain embodiments, the TGF-β type I receptor signaling inhibitor is A83-01, which is a potent inhibitor of ALK5 (type I transforming growth factor-β receptor), ALK4 (type IB activin receptor), and ALK7 (type I NODAL receptor) with an IC₅₀=12, 45, and 7.5 nM, respectively. According to some embodiments, the TGF-β type I receptor signaling inhibitor is present in the cell culture medium in an amount of from 0.1 to 10 μM, e.g., from 0.5 to 2 μM, from 0.5 to 1.5 μM, e.g., about 1 μM.

In certain embodiments, the cell culture medium comprises a cell-permeable glycogen synthase kinase 3 (GSK-3) inhibitor. GSK-3 is a serine/threonine protein kinase that phosphorylate either threonine or serine, and this phosphorylation controls a variety of biological activities, such as glycogen metabolism, cell signaling, and cellular transport. According to some embodiments, the cell-permeable GSK-3 inhibitor is CHIR99021. CHIR99021 is a selective small molecule GSK-3 inhibitor that activates Wnt signaling. See Ye at al. (2012) PLOS ONE 7(4): e35892. In certain embodiments, the GSK-3 inhibitor is present in the cell culture medium in an amount of from 0.1 to 10 μM, e.g., from 1 to 5 μM, such as about 3 μM.

According to some embodiments, the cell culture medium comprises Leukemia Inhibitory Factor (LIF). Leukemia inhibitory factor (LIF) is a cytokine which belongs to the IL-6 superfamily. LIF derives its name from its ability to inhibit proliferation of myeloid leukemia cells in culture. LIF signals via binding to the LIF receptor (LIFR-α), which forms a heterodimer with the glycoprotein 130. In certain embodiments, the LIF is recombinant LIF, e.g., recombinant mouse LIF. According to some embodiments, the LIF is chicken LIF. In some instances, the LIF (e.g., chicken LIF) is present in the cell culture medium in an amount of from 25 to 75 U/ml, e.g., from 45 to 55 U/ml, such as about 50 U/ml.

In certain embodiments, the cell culture medium comprises a cell-permeable Rho-associated, coiled-coil containing protein kinase (ROCK) inhibitor. Rho family proteins and their effectors are ubiquitously distributed and regulate various cell behaviors, including cell migration, cell adhesion to substrate, cell-to-cell fusion and apoptosis. According to some embodiments, the cell-permeable ROCK inhibitor is Y-27632. See Motomura et al. (2017) PLOS ONE 12(5): e0177994. In certain embodiments. the ROCK inhibitor is present in the cell culture medium in an amount of from 5 to 15 μM, e.g., from 8 to 12 μM, such as about 10 μM.

According to some embodiments, the cell culture medium comprises serum. In certain instances, the serum is chicken serum. When the cell culture medium comprises serum (e.g., chicken serum), in some embodiments, the serum is present in the cell culture medium at a concentration of from 5 to 15%, e.g. from 8 to 12%, such as about 10%.

As summarized above, the methods of the present disclosure comprise culturing the epiblast cells in a feeder-free cell culture container. As used herein, “feeder cells” or “feeders” are cells of one type that are co-cultured with cells of a second type, to provide an environment in which the cells of the second type can grow. By “feeder-free” cell culture container is meant a culture container wherein less than 10% of the total cells in the container are feeder cells, such as, e.g., less than 5%, less than 4%, less than 3%, less than 2%, less than 1%, less than 0.5%, less than 0.1%, less than 0.01%, or 0% (i.e., the container contains no feeder cells).

According to some embodiments, the feeder-free cell culture container is coated. For example, the cell culture container may be a glycoprotein-coated cell culture container. As used herein, a “glycoprotein” is a protein comprising glycans attached to amino acid side chains. In certain embodiments, the glycoprotein is of the hemopexin family of glycoproteins. A non-limiting example of a glycoprotein of the hemopexin family which may be employed according to the methods of the present disclosure is vitronectin (VTN-N), e.g., recombinant human VTN-N. Thus, according to some embodiments, the feeder-free cell culture container is a VTN-N-coated cell culture container. Details of an example approach for preparing VTN-N-coated cell culture containers suitable for use according to the present methods are described in the Experimental section below. Suitable cell culture containers include, e.g., cell culture plates, such as single-well or multi-well cell (e.g., 4-well, 8-well, 24-well, etc.) cell culture plates.

In certain embodiments, the culturing comprises culturing the epiblast cells in the feeder-free cell culture container to confluency, e.g., high confluency. Cell confluence is defined as the percentage of the surface area of two-dimensional (2D) culture that is covered with cells. According to some embodiments, the epiblast cells are cultured in the feeder-free cell culture container to a confluency of 75% or greater, 80% or greater, 85% or greater, 90% or greater, 95% or greater, or 100% confluency. In certain embodiments, the methods of the present disclosure comprise passaging the cells one or more times. The passaging may include, e.g., supplementation and/or replacement of the cell culture medium with a cell dissociation agent (e.g., an enzyme-free cell dissociation agent such as ReLeSR™ cell dissociation agent), picking of cells with the aid of a dissection microscope, and transferring colonies of the cells to new feeder-free cell culture containers.

According to some embodiments, the methods result in the production of pluripotent avian embryonic stem cells. As used herein, “pluripotent” avian embryonic stem cells refers to avian embryonic stem cells capable of differentiating into cells derived from any of the three germ layers: endoderm (interior stomach lining, gastrointestinal tract, the lungs), mesoderm (muscle, bone, blood, urogenital), or ectoderm (epidermal tissues and nervous system). An example feeder-free approach for producing pluripotent avian embryonic stem cells is described in detail in the Experimental section below.

Also provided are methods comprising genetically modifying avian embryonic stem cells produced according to the methods of the present disclosure. In certain embodiments, the genetic modification comprises ablating or deleting all or a portion of an endogenous gene or otherwise render non-functional one or more endogenous genes. Such deletion of a gene, or portion thereof, rendering the gene and/or the encoded product non-functional may be referred to as a knock-out. In some instances, a gene, or the gene product thereof, may be rendered non-functional through introduction of an insertion, e.g., causing a frameshift or generating a misfolded or otherwise non-functional protein. In some instances, multiple genetic modifications may be introduced into a single cell. For example, in some instances, a cell may include more than one deletion, insertion, substitution, or some combination thereof, including, e.g., where the cell include 2, 3, 4, or 5 such genetic modifications.

Various convenient methods of contacting a cell population with one or more reagents for genetic modification may be employed including but not limited to e.g., transfection of reagents and/or nucleic acids encoding such agents, transduction of genetic modification reagents, nucleofection and/or electroporation of genetic modification reagents, and the like. In some instances, a vector, e.g., a viral vector or a non-viral vector may be employed. In some instances, the components of the vector may include nucleic acids, proteins, or a combination thereof. Any convenient viral or non-viral vector may be employed including but not limited to e.g., lipid nanoparticle (LNP) vectors.

Vectors may be configured to contain all, or less than all, of the components necessary for performing a desired genetic modification. For example, in some instances, a vector may include all components sufficient for performing a genetic modification at a targeted locus. In some instances, a vector may include less than all of the components needed for performing a genetic modification and the remaining components may be delivered by other means, e.g., another different vector, transduction, transfection, or the like. In some instances, components, e.g., nucleic acid and protein components, of a targeting system may be pre-complexed prior to delivery, including where such components are pre-complexed within a delivery vector. For example, in some instances nucleic acid (e.g., a gRNA, etc.) and protein (e.g., nuclease(s) or base editing protein(s), etc.) editing reagents of an editing system may be complexed as ribonucleoprotein (RNP) for delivery to a cell population for genetic modification.

Any convenient and appropriate genetic modification system may be employed to introduce one or more of the genetic modifications described herein. Methods of site-directed introduction of a desired genetic modification will vary and may include introducing one or more site directed cleavage events, e.g., through the use of one or more site-directed nucleases (e.g., a CRISPR/Cas9 nuclease, a TALEN nuclease, a ZFN, and the like). Site-directed cleavage may include double and/or single strand breaks where applicable. In some instances, site-directed cleavage is followed by a specific repair event at the site cleaved by the site-directed nuclease, e.g., to introduce a desired edit, such as e.g., a substitution, insertion, deletion, or the like. Such methods of specific repair may include, e.g., homologous recombination, including homology directed repair (HDR), e.g., in the presence of a nucleic acid that includes homology regions to guide the repair. In some instances, site-directed cleavage may be employed to introduce a gene disruption and/or knock-out, e.g., without employing a specific repair event, e.g., through cellular processes following site-directed cleavage such as e.g., non-homologous end joining (NHEJ). In some instances, site-directed introduction of a desired genetic modification may employ a base editing system that does not introduce a double strand cleavage event, such as but not limited to e.g., CRISPR protein-guided based editing systems, such as e.g., dCas9-deaminase fusion protein systems including cytosine base editor (CBE) and adenine base editor (ABE) systems. In some instances, useful base editing systems introduce a single base change, e.g., without cleavage of the phosphodiester nucleic acid backbone.

Various genetic modification compositions may be employed and such compositions will vary, e.g., based on the genetic modification system employed, the type of genetic modification desired, the sequence of a targeted locus or loci, etc. Useful genetic modification compositions may include e.g., CRISPR/Cas9 editing compositions, e.g., including a Cas9 protein, or a nucleic acid encoding a Cas9 protein, and gRNAs or a sgRNA or a nucleic acid encoding the gRNAs or sgRNA; TALEN editing compositions, including e.g., a TALEN nuclease or TALEN nuclease pair, or a nucleic acid encoding a TALEN nuclease or TALEN nuclease pair; ZFN editing compositions, including e.g., a ZFN nuclease or ZFN nuclease pair, or a nucleic acid encoding a ZFN nuclease or ZFN nuclease pair; base-editing editing compositions e.g., including a CRISPR-protein-guided-base-editing protein, or a nucleic acid encoding a CRISPR-protein-guided-base-editing protein, and gRNAs or a sgRNA or a nucleic acid encoding the gRNAs or sgRNA; and the like.

According to some embodiments, useful genetic modification (sometimes referred to herein as “editing compositions”) will include a CRISPR-Cas protein, such as e.g., a Cas9 protein, or a polynucleotide encoding a CRISPR-Cas protein and guide RNA (gRNA) or a polynucleotide encoding gRNA. As used herein, the term “gRNA” generally encompasses either two-component guide systems (e.g., two gRNAs) as well as single guide RNA (sgRNA) systems, unless inappropriate and/or denoted otherwise. In some instances, the gRNA or multiple gRNAs may be configured and employed to target a desired locus as described herein or one or more elements thereof such as one of more exons of a gene present at the locus. For example, in some instances, a gRNA or multiple gRNAs may be configured and employed to target a locus or one or more elements thereof, such as e.g., one or more exons of the locus.

In certain embodiments, the genetic modification may include the use of a Cas9 nuclease, including natural and engineered Cas9 nucleases, as well as nucleic acid sequences encoding the same. Useful Cas9 nucleases include but are not limited to e.g., Streptococcus pyogenesCas9 and variants thereof, Staphylococcus aureus Cas9 and variants thereof, Actinomyces naeslundii Cas9 and variants thereof, Cas9 nucleases also include those discussed in PCT Publications Nos. WO 2013/176772 and WO2015/103153 and those reviewed in e.g., Makarova et al. (2011) Nature Reviews Microbiology 9:467-477, Makarova et al. (2011) Biology Direct 6:38, Haft et al. (2005) PLOS Computational Biology 1: e60 and Chylinski et al. (2013) RNA Biology 10:726-737, the disclosures of which are incorporated herein by reference in their entirety. In some instances, a non-Cas9 CRISPR nuclease (or engineered variant thereof) may be employed, including but not limited to e.g., Cpf1 or Cpf1 variant.

The CRISPR system offers significant versatility in gene editing in part because of the small size and high frequency of necessary sequence targeting elements within host genomes. CRISPR guided Cas9 nuclease requires the presence of a protospacer adjacent motif (PAM), the sequence of which depends on the bacteria species from which the Cas9 was derived (e.g. for Streptococcus pyogenes the PAM sequence is “NGG”) but such sequences are common throughout various target nucleic acids. The PAM sequence directly downstream of the target sequence is not part of the guide RNA but is obligatory for cutting the DNA strand. Synthetic Cas9 nucleases have been generated with novel PAM recognition, further increasing the versatility of targeting, and may be used in the methods described herein. Cas9 nickases (e.g., Cas9 (D10A) and the like) that cleave only one strand of target nucleic acid as well as endonuclease deficient (i.e., “dead”) dCas9 variants with additional enzymatic activities added by an attached fusion protein have also been developed.

In certain embodiments, a method of genetic modification may include the use of a zinc-finger nuclease (ZFN). ZFNs consist of the sequence-independent Fokl nuclease domain fused to zinc finger proteins (ZFPs). ZFPs can be altered to change their sequence specificity. Cleavage of targeted dsDNA involves binding of two ZFNs (designated left and right) to adjacent half-sites on opposite strands with correct orientation and spacing, thus forming a Fokl dimer. Dimerization increases ZFN specificity significantly. Three or four finger ZFPs target about 9 or 12 bases per ZFN, or about 18 or 24 bases for the ZFN pair. The specificity, efficiency and versatility of targeting and replacement of homologous recombination is greatly improved through the combined use of various homology-directed repair strategies and ZFNs (see e.g., Urnov et al. (2005) Nature. 435(7042):646-5; Beumer et al (2006) Genetics. 172(4):2391-2403; Meng et al (2008) Nat Biotechnol. 26(6):695-701; Perez et al. (2008) Nat Biotechnol. 26(7):808-816; Hockemeyer et al. (2009) Nat Biotechnol. 27(9):851-7; the disclosures of which are incorporated herein by reference in their entirety). In general, one ZFN site can be found every 125-500 bp of a random genomic sequence, depending on the assembly method. Methods for identifying appropriate ZFN targeting sites include computer-mediated methods e.g., as described in e.g., Cradick et al. (2011) BMC Bioinformatics. 12:152, the disclosure of which is incorporated herein by reference in its entirety.

According to some embodiments, a method of genetic modification may include the use of a transcription activator-like effector nuclease (TALEN). Similar in principle to the ZEN nucleases, TALENs utilize the sequence-independent Fokl nuclease domain fused to Transcription activator-like effectors (TALEs) proteins that, unlike ZNF, individually recognize single nucleotides. TALEs generally contain a characteristic central domain of DNA-binding tandem repeats, a nuclear localization signal, and a C-terminal transcriptional activation domain. A typical repeat is 33-35 amino acids in length and contains two hypervariable amino acid residues at positions 12 and 13, known as the “repeat variable di-residue” (RVD). An RVD is able to recognize one specific DNA base pair and sequential repeats match consecutive DNA sequences. Target DNA specificity is based on the simple code of the RVDs, which thus enables prediction of target DNA sequences. Native TALEs or engineered/modified TALEs may be used in TALENs, depending on the desired targeting. TALENs can be designed for almost any sequence stretch. Merely the presence of a thymine at each 5′ end of the DNA recognition site is required. The specificity, efficiency and versatility of targeting and replacement of homologous recombination is greatly improved through the combined use of various homology-directed repair strategies and TALENs (see e.g., Zu et al. (2013) Nature Methods. 10:329-331; Cui et al. (2015) Scientific Reports 5:10482; Liu et al. (2012) J. Genet. Genomics. 39:209-215, Bedell et al. (2012) Nature. 491:114-118, Wang et al. (2013) Nat. Biotechnol. 31:530-532; Ding et al. (2013) Cell Stem Cell. 12:238-251; Wefers et al. (2013) Proc. Natl. Acad. Sci. U.S.A, 110:3782-3787; the disclosures of which are incorporated herein by reference in their entirety).

In certain embodiments, a method of genetic modification may include the use of a base editor system, including but not limited to e.g., base editor systems employing a fusion protein comprising a programable DNA binding protein, a nucleobase editor and gRNA, and the like. Base editing will generally not rely on HDR and/or NHEJ and will generally not result in or require the cleavage of phosphodiester bonds on both backbones of dsDNA. Thus, based editing may, in some instances, employ RNA-guided (i.e., “programable”) DNA binding proteins, such as Cas nucleases, that do not cause double-strand breaks, such as e.g., nuclease-deficient or nuclease-defective Cas proteins, such as e.g., a dCas9 or a Cas9 nickase. Useful examples of base editors and base editing systems, including base editor encoding nucleic acids, include but are not limited to BE1, BE2, BE3 (Komor et al., 2016); Target-AID (Nishida et al., 2016); SaBE3, BE3 PAM variants, BE3 editing window variants (Kim et al., 2017); HF-BE3 (Rees et al., 2017); BE4 and BE4-Gam; AID, CDA1 and APOBEC3G BE3 variants (Komor et al., 2017); BE4max, ArcBe4max, ABEmax (Koblan et al., 2018); Adenine base editors (ABE7.10) (Gaudelli et al., 2017); ABE8 (Richter et al., 2020); ABE8e (Gaudelli et al., 2020); A&C-BEmax (Zhang et al., 2020); SPACE (Grünewald et al., 2020); and the like; the preceding references being incorporated by reference herein in their entirety.

Other useful components, e.g., of transgenes, of expression cassettes, of editing compositions, of vectors, or the like, may include promoter sequences (e.g., constitutive, tissue-specific, etc.), signal peptide sequences, poly (A) sequences, terminators, translational regulatory sequences such as ribosome binding sites and internal ribosome entry sites, enhancers, silencers, insulators, boundary elements, replication origins, matrix attachment sites and/or locus control regions. Furthermore, multiple gene products can be expressed from one nucleic acid, for example by linking individual components (transgenes) in one open reading frame separated, for example, by a self-cleaving 2A peptide or IRES sequence.

Examples of useful promoters include, for example, viral simian virus 40 (SV40) (e.g., early or late), cytomegalovirus (CMV) (e.g., immediate early), Moloney murine leukemia virus (MoMLV), MND (myeloproliferative sarcoma virus enhancer, negative control region deleted, dl587rev primer-binding site substituted), Rous sarcoma virus (RSV), herpes simplex virus (HSV), spleen focus-forming virus (SFFV) promoters and the like. In certain embodiments, the promoter may be inducible, such that transcription of all or part of the viral genome will occur only when one or more induction factors are present. Induction factors include, but are not limited to, one or more chemical compounds or physiological conditions, e.g., temperature or pH, in which the host cells are cultured. In some instances, the promoter may be constitutive. In some instances, the promoter may cause preferential expression in a desired cell-type or tissue, e.g., the promoter may be cell-type or tissue specific.

Vectors, including retroviral vectors, e.g., lentivirus vectors, may include (or exclude as desired where appropriate) various elements, including cis-acting elements, such as promoters, long terminal repeats (LTR), and/or elements thereof, including 5′ LTRs and 3′ LTRs and elements thereof, central polypurine tract (cPPT) elements, DNA flap (FLAP) elements, export elements (e.g., rev response element (RRE), hepatitis B virus post-transcriptional regulatory element (HPRE), etc.), posttranscriptional regulatory elements (e.g., woodchuck hepatitis virus posttranscriptional regulatory element (WPRE), hepatitis B virus regulatory element (HPRE), etc.), polyadenylation sites, transcription termination signals, insulators elements (e.g., β-globin insulator, e.g., chicken HS4), and the like.

Functional integration of a transgene may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the targeted cells in a suitable transduction medium, at a suitable concentration (or multiplicity of infection), and for a suitable time for the vector to infect the target cells, facilitating functional integration of the transgene. By “functionally integrated”, as used herein, is generally meant that the transgene is integrated into the genome of the cell in such a way that the encoded gene product is expressed. Expression of the encoded gene product may be controlled, in whole or in part, by endogenous components of the cell or exogenous (including heterologous) components included in the transgene. For example, in some instances, expression of the encoded gene product may be controlled by one or more endogenous regulatory elements, e.g., promoter, enhancer, etc., at or near the genomic locus into which the transgene is inserted. In some instances, expression of the encoded gene product may be controlled by one or more exogenous (including heterologous) regulatory elements, e.g., promoter, enhancer, etc., present in the transgene, and operably linked to the encoded gene product, prior to insertion.

In certain embodiments, the genetically modifying comprises inactivating one or more endogenous genes of the avian embryonic stem cells. For example, the genetically modifying may comprise inactivating (e.g., knocking out) one or more of the endogenous genes, and/or transiently or permanently downregulating a target gene, e.g., via RNA interference, morpholino, and/or the like. Approaches for making gene knockouts and downregulating genes are known and described herein.

According to some embodiments, the genetically modifying comprises transiently or permanently overexpressing a gene. In certain embodiments, the genetically modifying comprises introducing one or more transgenes into the avian embryonic stem cells. Details of approaches for introducing transgenes into cells are described above. In some instances, the one or more transgenes comprise an avian transgene. In some instances, the one or more transgenes comprise a non-avian transgene. Non-avian transgenes of interest include those that encode a wild-type or mutated mammalian (e.g., primate, such as human) or reptilian protein. Non-avian transgenes of interest also include those that encode a nature-derived or synthetic protein. For example, in some instances, the non-avian transgene encodes a biosensor protein. Non-limiting examples of biosensor proteins include GCaMP6, jGCaMP7, jGCaMP8, ASAP1, ASAP2, QuasAr1, QuasAr2, QuasAr3, GRAB-DA, GRAB-DA2, GRAB5-HT, GRABeCB2, GRABNE, iAChSnFR, GRABAch, iSeroSnFR, iGABASnFR, pHluorin, and pHluorin2. Also by way of example, in some instances, the non-avian transgene encodes an actuator protein. Non-limiting examples of actuator proteins include ChR2, bReaChES, Chrimson, ChRmine, CheRiff, ArchT, eOPN3, eArch3.0, and Arch. According to some embodiments, the non-avian transgene encodes an endonuclease protein. Non-limiting examples of endonuclease proteins include Cas proteins, e.g., Cas9, Cas13, Cas12, Cas14, CasMINI, or the like. In certain embodiments, the endonuclease protein is a catalytically inactive endonuclease protein (i.e., an endonuclease protein which lacks endonuclease activity). For example, the catalytically inactive endonuclease protein may be a catalytically inactive Cas protein, non-limiting examples of which include dead Cas9 (dCas9) or dead Cas13 (dCas13).

### AVIAN EMBRYONIC STEM CELLS AND METHODS OF USE

Also provided are avian embryonic stem cells produced according to the methods of the present disclosure. Compositions comprising such cells are also provided. Harvested avian embryonic stem cells produced by the methods as described herein may be present in any suitable container (e.g., a culture vessel, tube, flask, vial, cryovial, cryo-bag, etc.) and may be employed (e.g., differentiated into a cell, organoid, tissue, etc. and/or engrafted into a non-human animal) using any suitable methods and/or devices. Such a population of avian embryonic stem cells may be prepared and/or used fresh or may be cryopreserved. In some instances, populations of avian embryonic stem cells may be prepared in a “ready-to-use” format, including e.g., where the cells are present in a suitable diluent and/or at a desired concentration (e.g., for differentiation into a cell, organoid, tissue, etc. and/or engraftment into a non-human animal).

In certain embodiments, the compositions may include the avian embryonic stem cells present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, glycerol, a chelating agent, and the like may be present in such compositions.

According to some embodiments, the avian embryonic stem cells are cryopreserved. As used herein, “cryopreserved” refers to cells that have been preserved or maintained by cooling to low sub-zero temperatures, such as 77 K or −196 deg. C. (the boiling point of liquid nitrogen). At these low temperatures, any biological activity, including the biochemical reactions that would lead to cell death, is effectively stopped. Useful methods of cryopreservation and thawing cryopreserved cells, as well as processes and reagents related thereto, include but are not limited to e.g., those described in U.S. Pat. Nos. 10,370,638; 10159244; 9078430; 7604929; 6136525; and 5795711, the disclosures of which are incorporated herein by reference in their entirety. In contrast, the term “fresh”, as used herein with reference to cells, may refer to avian embryonic stem cells that have not been cryopreserved.

In certain embodiments, for cryopreservation, a cell suspension is aliquoted into one or more vessels and pelleted by centrifugation. Cell pellets may then be resuspended in cryopreservation media under cold conditions to reach a desired final concentration, such as e.g., 10 million live cells per mL, and the resuspended cells kept at 4-8 deg. C. Cells prepared for cryopreservation may then be aliquoted into freezing containers and frozen using a controlled rate freezer. After controlled rate freezing is complete, cryopreserved may then be transferred to vapor phase liquid nitrogen for storage.

According to some embodiments, the avian embryonic stem cells are infected by a virus. Non-limiting examples of viruses by which the avian embryonic stem cells may be infected include a lentivirus, an adenovirus, an adeno-associated virus, a retrovirus, a coronavirus, and a herpesvirus.

Aspects of the present disclosure include methods of using the avian embryonic stem cells of the present disclosure. In certain embodiments, such methods comprise producing a two-dimension culture, a spheroid, or an organoid from avian embryonic stem cells produced according to the methods of the present disclosure. By “organoid” is meant a three-dimensional (3D) multicellular in vitro or ex vivo tissue construct that may mimic a corresponding in vivo organ.

According to some embodiments, provided are methods of producing an avian telencephalic organoid from avian embryonic stem cells produced according to the methods of the present disclosure. In certain embodiments, such methods comprise culturing the avian embryonic stem cells in a telencephalon differentiation medium. In some instances, the telencephalon differentiation medium comprises a cell-permeable ROCK inhibitor, a non-limiting example of which includes Y-27632. The ROCK inhibitor may be present in the telencephalon differentiation medium in an amount of from 5 to 15 μM, e.g., from 8 to 12 μM, such as about 10 μM.

In some instances, the telencephalon differentiation medium comprises a WNT inhibitor. The Wnt signaling pathway is a conserved pathway in metazoan animals. The extra-cellular Wnt signal stimulates several intra-cellular signal transduction cascades, including the canonical or Wnt/β-catenin dependent pathway and the non-canonical or β-catenin-independent pathway which can be divided into the Planar Cell Polarity pathway and the Wnt/Ca²⁺pathway. In certain embodiments, WNT inhibitor is IWR1-ε, which inhibits WNT signaling by blocking a cell-based WNT/β-catenin pathway reporter response. It inhibits WNT-induced accumulation of β-catenin, through stabilization of the destruction complex member AXIN2. According to some embodiments, the WNT inhibitor is present in the telencephalon differentiation medium in an amount of from 1 to 10 μM, e.g., from 1 to 5 M, such as about 3 μM.

In certain embodiments, the telencephalon differentiation medium comprises a TGF-β pathway inhibitor. A non-limiting example of a TGF-β pathway inhibitor which finds use in the telencephalon differentiation medium is SB431542, a selective and potent inhibitor of the TGF-β/Activin/Nodal pathway that inhibits ALK5, ALK4, and ALK7. According to some embodiments, the TGF-β pathway inhibitor is present in the telencephalon differentiation medium in an amount of from 1 to 10 μM, e.g., 3 to 7 μM, such as about 5 μM.

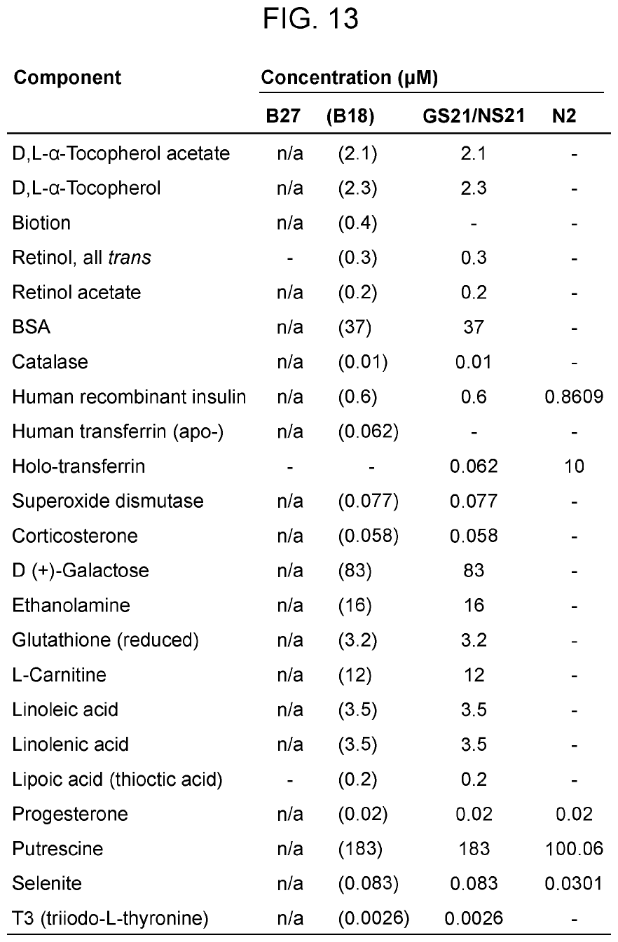

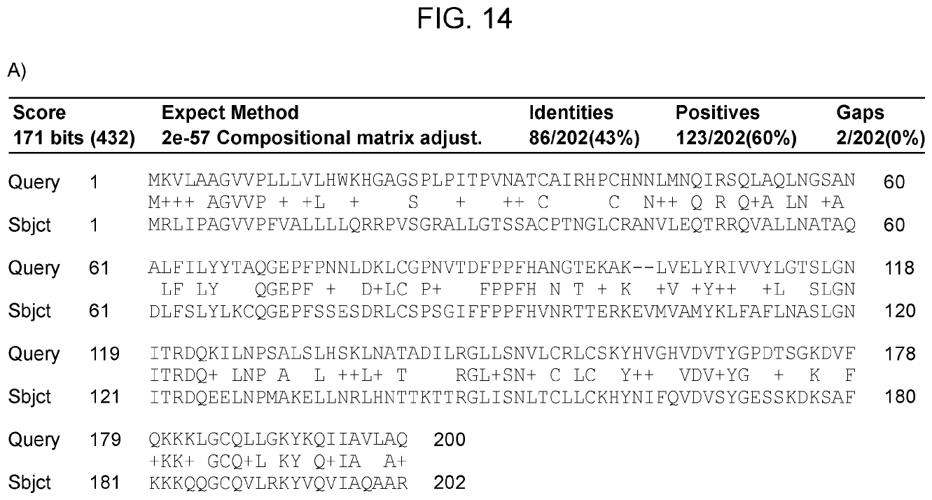

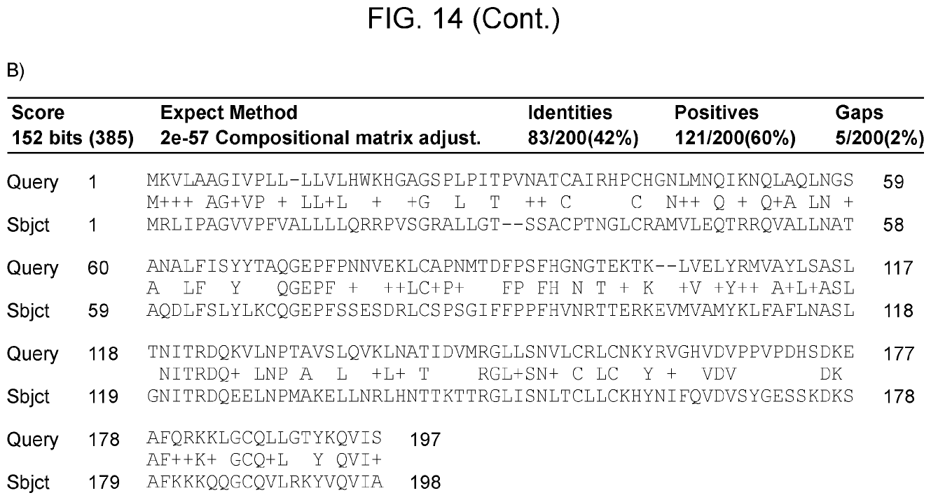

According to some embodiments, the cells are cultured in the telencephalon differentiation medium for 5 to 9 days, e.g., 6 to 8 days, such as about 7 days. In some instances, subsequent to the culturing in the telencephalon differentiation medium, the methods of producing avian telencephalic organoids further comprise culturing the produced organoids in a neuronal differentiation medium. In certain embodiments, the neuronal differentiation medium comprises N-2 supplement. A non-limiting example of an N-2 supplement formulation is provided in FIG. 13. According to some embodiments, subsequent to the culturing in the neuronal differentiation medium (e.g., for 15 to 35 days, such as for 20 to 30 days, e.g., about 25 days), the methods of producing avian telencephalic organoids further comprise culturing the produced organoids in a neuronal maturation medium. The neuronal maturation medium may comprise, e.g., N-2 supplement, B-27 supplement, and BrainPhys component.

As demonstrated in the Experimental section below, avian telencephalic organoids produced according to the methods of the present disclosure contain pS6, a downstream target of the mTOR pathway, which is a highly conserved in the dorsal pallium of the telencephalon. They also contain the presence of neurons and astroglial cells. A subset of the neurons express the neuronal maturation marker Mef2c, and a subset of neurons express SATB2 which specifically labels neurons of dorsal pallium in the avian telencephalon. Thus, the organoids produced according to the instant methods contain a diverse set of cells of telencephalic fate, including maturing and mature neurons of the dorsal pallium, as well as astroglia cells.

Aspects of the present disclosure further include spheroids and organoids produced according to the instant methods.

Also provided by the present disclosure are methods comprising engrafting into a non-human animal the avian embryonic stem cells, spheroids, and/or organoids of the present disclosure. The non-human animal may vary. In some instances, the non-human animal is an avian animal (e.g., a chicken, etc.). According to some embodiments, the non-human animal is mammal, a non-limiting example of which is a rodent (e.g., mouse or rat). The non-human animal may be fully developed (e.g., an adult non-human animal) at the time of engraftment, or the engraftment may occur when the non-human animal is developing, e.g., at an embryonic, fetal, neo-natal, juvenile, adolescent, or young adult stage of development. Also provided by the present disclosure are methods comprising engrafting into a fertilized egg or an unfertilized egg the avian embryonic stem cells of the present disclosure.

The avian embryonic stem cells, spheroids and organoids of the present disclosure find use in a variety of contexts, including but not limited to, methods of screening of test (e.g., candidate) agents. Test agents of interest include peptides, polypeptides, ligands, small molecules, aptamers, immunogenic agents (e.g., vaccines), and the like. The terms “polypeptide”, “peptide”, or “protein” are used interchangeably herein to designate a linear series of amino acid residues connected one to the other by peptide bonds between the alpha-amino and carboxy groups of adjacent residues. The amino acids may include the 20 “standard” genetically encodable amino acids, amino acid analogs, or a combination thereof. As used herein, a “ligand” is a substance that forms a complex with a biomolecule to serve a biological purpose. The ligand may be a substance selected from, e.g., a circulating factor, a secreted factor, a cytokine, a growth factor, a hormone, a peptide, a polypeptide, a small molecule, and a nucleic acid, that forms a complex with a cell surface molecule on the surface of a cell. By “small molecule” is meant a compound having a molecular weight of 1000 atomic mass units (amu) or less. In some embodiments, the small molecule is 900 amu or less, 750 amu or less, 500 amu or less, 400 amu or less, 300 amu or less, or 200 amu or less. In certain aspects, the small molecule is not made of repeating molecular units such as are present in a polymer. By “aptamer” is meant a nucleic acid (e.g., an oligonucleotide) that has a specific binding affinity for the target cell surface molecule.

According to some embodiments, a screening method of the present disclosure comprises contacting avian embryonic stem cells produced according to the methods of the present disclosure with a test agent, and assessing for an effect (e.g., a biological effect) of the test agent on the avian embryonic stem cells. In other embodiments, a screening method of the present disclosure comprises contacting spheroids or organoids produced according to the methods of the present disclosure with a test agent, and assessing for an effect (e.g., a biological effect) of the test agent on the spheroids or organoids. In some embodiments, avian telencephalic organoids produced according to the methods of the present disclosure are employed in the screening methods. Organoids recapitulate many biological parameters of tissue development, such as the organization of heterogeneous cells and cell-cell/cell-matrix interactions. Compared with 2D cultures and in vivo models, organoids are more amenable to the manipulation of stem cell niche components and genome editing. The versatile nature of organoid models facilitate a range of biomedical applications, including investigations of tissue renewal, organ development, disease etiology, viral infection, and drug discovery. The utility and approaches for utilizing organoids in drug screening, for example, is described in Calandrini et al. (2021) Cell Reports 36(8) 109568; Takahashi (2019) Annual Review of Pharmacology and Toxicology 59:447-462; and elsewhere.

Notwithstanding the appended claims, the present disclosure is also defined by the following embodiments.
1. A method of producing avian embryonic stem cells, the method comprising: culturing epiblast cells obtained from a stage IX-XI avian embryo in a cell culture medium present in a feeder-free cell culture container.
2. The method according to embodiment 1, wherein the cell culture medium comprises active FGF-2.
3. The method according to embodiment 1 or embodiment 2, wherein the cell culture medium comprises a cell-permeable MEK/ERK pathway inhibitor.
4. The method according to embodiment 3, wherein the cell-permeable MEK/ERK pathway inhibitor is PD0325901.
5. The method according to embodiment 3 or embodiment 4, wherein the cell-permeable MEK/ERK pathway inhibitor is present in an amount of from 0.01 to 1 μM, optionally from 0.05 to 0.5 M.
6. The method according to any one of embodiments 1 to 5, wherein the cell culture medium comprises a TGF-beta type I receptor signaling inhibitor.
7. The method according to embodiment 6, wherein the TGF-beta type I receptor signaling inhibitor is A83-01.
8. The method according to embodiment 6 or embodiment 7, wherein the TGF-beta type I receptor signaling inhibitor is present in an amount of from 0.1 to 10 μM, optionally from 0.5 to 2μM.
9. The method according to any one of embodiments 1 to 8, wherein the cell culture medium comprises a cell-permeable glycogen synthase kinase 3 (GSK-3) inhibitor.
10. The method according to embodiment 9, wherein the cell-permeable GSK-3 inhibitor is CHIR99021.
11. The method according to embodiment 9 or embodiment 10, wherein the GSK-3 inhibitor is present in an amount of from 0.1 to 10 μM, optionally from 1 to 5 μM.
12. The method according to any one of embodiments 1 to 11, wherein the cell culture medium comprises Leukemia Inhibitory Factor (LIF).
13. The method according to embodiment 12, wherein the LIF is chicken LIF.
14. The method according to embodiment 12 or 13, wherein the LIF is present in an amount of from 25 to 75 U/ml, optionally from 45 to 55 U/ml.
15. The method according to any one of embodiments 1 to 14, wherein the cell culture medium comprises a cell-permeable Rho-associated, coiled-coil containing protein kinase (ROCK) inhibitor.
16. The method according to embodiment 15, wherein the cell-permeable ROCK inhibitor is Y-27632.
17. The method according to embodiment 15 or embodiment 16, wherein the cell-permeable ROCK inhibitor is present in an amount of from 5 to 15 μM, optionally from 8 to 12 μM.
18. The method according to any one of embodiments 1 to 17, wherein the cell culture medium comprises chicken serum.
19. The method according to embodiment 18, wherein the chicken serum is present in an amount of from 5 to 15%, optionally from 8 to 12%.
20. The method according to any one of embodiments 1 to 19, wherein the feeder-free cell culture container is a glycoprotein-coated cell culture container.
21. The method according to embodiment 20, wherein the feeder-free cell culture container is a vitronectin (VTN-N)-coated cell culture container.
22. The method according to any one of embodiments 1 to 21, wherein prior to the culturing, the method comprises obtaining the epiblast cells from a stage IX-XI avian embryo.
23. The method according to embodiment 22, wherein the obtaining comprises removing the epiblast cells from a stage IX-XI avian embryo and washing the epiblast cells one or more times.
24. The method according to any one of embodiments 1 to 23, wherein the culturing comprises culturing the epiblast cells in the feeder-free cell culture container to confluency.
25. The method according to embodiment 24, further comprising passaging the cells.
26. The method according to any one of embodiments 1 to 25, wherein the avian embryo is a stage X avian embryo.
27. The method according to any one of embodiments 1 to 26, wherein the avian embryo is a chicken embryo.
28. The method according to any one of embodiments 1 to 27, wherein the avian embryonic stem cells are pluripotent avian embryonic stem cells.
29. A method comprising genetically modifying avian embryonic stem cells produced according to the methods of any one of embodiments 1 to 28.
30. The method according to embodiment 29, wherein the genetically modifying comprises inactivating one or more endogenous genes of the avian embryonic stem cells.
31. The method according to embodiment 30, wherein the inactivating comprises knocking out one or more of the endogenous genes.
32. The method according to any one of embodiments 29 to 31, wherein the genetically modifying comprises transiently or permanently downregulating a target gene.
33. The method according to any one of embodiments 29 to 32, wherein the genetically modifying comprises transiently or permanently overexpressing a gene.
34. The method according to any one of embodiments 29 to 33, wherein the genetically modifying comprises introducing one or more transgenes into the avian embryonic stem cells.
35. The method according to embodiment 34, wherein the one or more transgenes comprise an avian transgene.
36. The method according to embodiment 34 or embodiment 35, wherein the one or more transgenes comprise a non-avian transgene.
37. The method according to embodiment 36, wherein the non-avian transgene encodes a wild-type or mutated mammalian or reptilian protein.
38. The method according to embodiment 37, wherein the non-avian transgene encodes a wild-type or mutated human protein.
39. The method according to embodiment 36, wherein the non-avian transgene encodes a nature-derived or synthetic protein.
40. The method according to embodiment 36, wherein the non-avian transgene encodes a biosensor protein.
41. The method according to embodiment 40, wherein the biosensor protein is GCaMP6, jGCaMP7, jGCaMP8, ASAP1, ASAP2, QuasAr1, QuasAr2, QuasAr3, GRAB-DA, GRAB-DA2, GRAB5-HT, GRABeCB2, GRABNE, iAChSnFR, GRABAch, iSeroSnFR, iGABASnFR, pHluorin, or pHluorin2.
42. The method according to embodiment 36, wherein the non-avian transgene encodes an actuator protein.
43. The method according to embodiment 42, wherein the actuator protein is ChR2, bReaChES, Chrimson, ChRmine, CheRiff, ArchT, eOPN3, eArch3.0, or Arch.
44. The method according to embodiment 36, wherein the non-avian transgene encodes an endonuclease protein.
45. The method according to embodiment 44, wherein the endonuclease protein is a Cas protein.
46. The method according to embodiment 45, wherein the Cas protein is Cas9, Cas13, Cas12, Cas14, or CasMINI.
47. The method according to embodiment 44, wherein the endonuclease protein is a catalytically inactive endonuclease protein.
48. The method according to embodiment 47, wherein the catalytically inactive endonuclease protein is a catalytically inactive Cas protein.
49. The method according to embodiment 48, wherein the catalytically inactive Cas protein is dead Cas9 (dCas9) or dead Cas13 (dCas13).
50. Avian embryonic stem cells produced according to the method of any one of embodiments 1 to 49.
51. The avian embryonic stem cells of embodiment 50, wherein the avian embryonic stem cells are cryopreserved.
52. The avian embryonic stem cells of embodiment 50 or embodiment 51, wherein the avian embryonic stem cells are infected by a virus.
53. The avian embryonic stem cells of embodiment 52, wherein the virus is a lentivirus, an adenovirus, an adeno-associated virus, a retrovirus, a coronavirus, or a herpesvirus.
54. A method comprising differentiating the avian embryonic stem cells of any one of embodiments 50 to 53 into cells of an organ.
55. Cells of an organ produced according to the method of embodiment 54.
56. A method comprising producing a two-dimension culture, a spheroid, or an organoid from the avian embryonic stem cells of any one of embodiments 50 to 53.
57. The method according to embodiment 56, wherein the method comprises producing an organoid from the avian embryonic stem cells.
58. The method according to embodiment 57, wherein the organoid is an avian telencephalic organoid.
59. The method according to embodiment 58, wherein the method comprises culturing the avian embryonic stem cells in a telencephalon differentiation medium.
60. The method according to embodiment 59, wherein the telencephalon differentiation medium comprises a cell-permeable ROCK inhibitor.
61. The method according to embodiment 60, wherein the cell-permeable ROCK inhibitor is Y-27632.
62. The method according to embodiment 60 or embodiment 61, wherein the cell-permeable ROCK inhibitor is present in an amount of from 5 to 15 μM, optionally from 8 to 12 μM.
63. The method according to any one of embodiments 59 to 62, wherein the telencephalon differentiation medium comprises a WNT inhibitor.
64 The method according to embodiment 63, wherein the WNT inhibitor is IWR1-ε.
65. The method according to embodiment 63 or embodiment 64, wherein the WNT inhibitor is present in an amount of from 1 to 10 μM, optionally from 1 to 5 M.
66. The method according to any one of embodiments 59 to 65, wherein the telencephalon differentiation medium comprises a TGF-β pathway inhibitor.
67. The method according to embodiment 66, wherein the TGF-β pathway inhibitor is SB431542.
68. The method according to embodiment 66 or embodiment 67, wherein the TGF-β pathway inhibitor is present in an amount of from 1 to 10 μM, optionally from 3 to 7 μM.
69. The method according to any one of embodiments 58 to 68, wherein the culturing in the telencephalon differentiation medium is for 5 to 9 days, 6 to 8 days, or about 7 days.
70. The method according to any one of embodiments 58 to 69, further comprising culturing the produced organoid in a neuronal differentiation medium.
71. The method according to embodiment 70, wherein the neuronal differentiation medium comprises N-2 supplement.
72. The method according to embodiment 70 or 71, further comprising, subsequent to the culturing in the neuronal differentiation medium, culturing the produced organoid in a neuronal maturation medium.
73. The method according to embodiment 72, wherein the neuronal maturation medium comprises N-2 supplement, B-27 supplement, and BrainPhys component.
74. A spheroid or organoid produced according to the method of any one of embodiments 56 to 72.
75. The spheroid or organoid of embodiment 74, wherein the spheroid or organoid is cryopreserved.
76. A method comprising engrafting into a non-human animal the avian embryonic stem cells of any one of embodiments 50 to 53.
77. A method comprising engrafting into a non-human animal the cells of an organ of embodiment 55.
78. A method comprising engrafting into a non-human animal the spheroid or organoid of embodiment 74 or embodiment 75.
79. The method according to any one of embodiments 76 to 78, wherein the non-human animal is a developing non-human animal.
80. A method comprising engrafting into a fertilized egg or an unfertilized egg the avian embryonic stem cells of any one of embodiments 50 to 53.
81. A screening method comprising:
   contacting avian embryonic stem cells produced according to the method of any one of embodiments 1 to 49 with a test agent; and
   assessing for an effect of the test agent on the avian embryonic stem cells.
82. A screening method comprising:
   contacting spheroids or organoids produced according to the method of any one of embodiments 56 to 72 with a test agent; and
   assessing for an effect of the test agent on the spheroids or organoids.
83. The method according to embodiment 82, wherein an avian telencephalic organoid produced according to the method of any one of embodiments 58 to 72 is contacted with the test agent.

The following examples are offered by way of illustration and not by way of limitation.

### EXPERIMENTAL

### Example 1—Isolation of Avian Embryonic Stem Cells

Fertilized chicken (Gallus gallus) eggs were obtained immediately after ovopositioning and processed. All experimental procedures were performed within 2 hours following ovopositioning, which represents stage IX-XI of avian development (Bakst et al. (1997) Poultry Science 76(1):83-90).

Individual eggs were disinfected with 70% ethanol and wiped clean. Each egg was then broken up carefully, separating the yolk from the shell and transferred to a tissue culture grade petri dish. After manipulating the orientation of the yolk so that the blastocyst is face up, a 1 ml syringe filled with sterile 1X Phosphate-buffered saline (PBS) pH 7.4 (Thermo Fisher Scientific # 70011044) was used to puncture the fertilized egg through the area pellucida. Suction was used to remove the epiblasts and transfer the cells to a 15 ml centrifuge tube filled with 10 ml of Stem Flex media (Thermo Fisher Scientific #A3349401). The samples were then centrifuged at 300 g for 3 minutes, followed by removal of the supernatant. The washes were repeated 3 times.

Tissue culture 24 well plates were coated with 500 ng/ml truncated human Vitronectin (VTN-N) (Thermo Fisher Scientific #A14700) dissolved in UltraPure DNase/RNase-Free Distilled Water (Thermo Fisher Scientific #10977015) for 15 minutes at room temperature and then washed 3 times with UltraPure Water.

The cells were transferred to the VTN-N coated plates and cultured in Stem Flex medium supplemented with 0.1 μm PD0325901 (Stem Cell Technologies #72182), 1 μm A83-01 (Stem Cell Technologies #72022), 3 μm CHIR99021 (Stem Cell Technologies #72054), 50 U/ml chicken leukemia inhibitory factor protein (chicken LIF) (Kingfisher Microscope #RP1395C-100), and 10 μm Y-27632 dihydrochloride. Media was changed after 12 hours.

### Example 2—Maintenance and Passaging of Avian Embryonic Stem Cells

Cells were cultured in VTN-N coated tissue culture plates containing Stem Flex medium supplemented with 10% Chicken Serum (Thermo Fisher Scientific #16110082), 0.1 μm PD0325901 (Stem Cell Technologies #72182), 1 μm A83-01 (Stem Cell Technologies #72022), 3 μm CHIR99021 (Stem Cell Technologies #72054) and 50 U/ml chicken LIF (Kingfisher Microscope #RP1395C-100). Media was changed daily.

When the cells reached high confluency, they were passages as follows: the media was removed from the cells and replaced with ReLeSR reagent (Stem Cell Technologies #100-0484) for 2 minutes. ReLeSR was removed and carefully replaced with Stem Flex medium. Cells were then manually picked with the aid of a dissection microscope (AmScope #SM-1TG-V331) and colonies of cells were transferred to new VTN-N coated tissue culture plates. The colonies were pipetted vigorously to break them into smaller aggregates.

Images of colonies of the chicken pluripotent embryonic stem cells (a cell line designated “HE2M38”) are provided in FIG. 3.

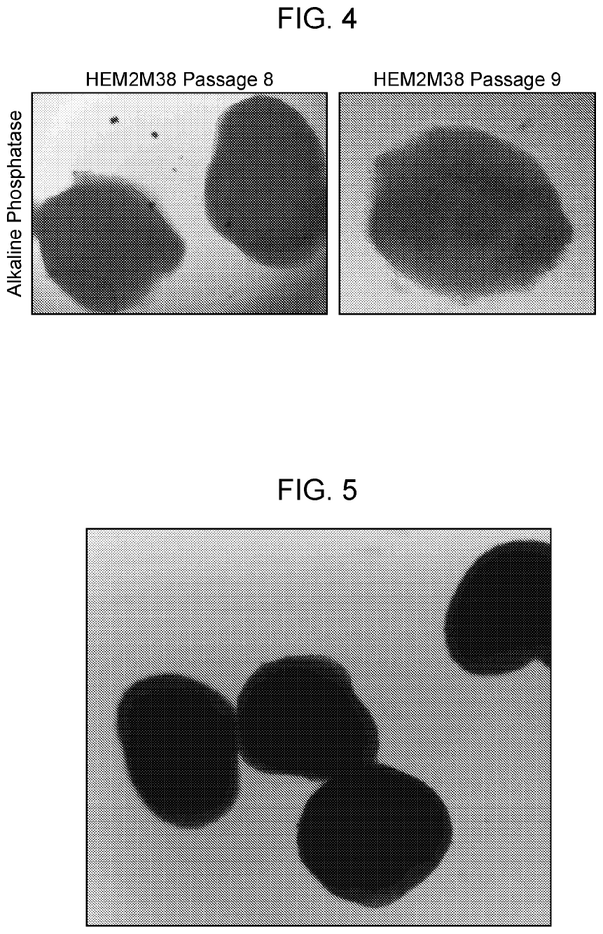

Images showing alkaline phosphatase staining of the HE2M38 chicken pluripotent embryonic stem cells are provided in FIG. 4.

### Example 3—Generation of Avian Telencephalic Organoids

Avian telencephalic organoids were generated by adapting previous protocols for derivation of mammalian cortical organoids (Eiraku et al. (2008) Cell Stem Cell 3(5):519-532). The embryonic stem cells were incubated in ReLeSR dissociation reagent (Stem Cell Technologies #05872) for 3 minutes, followed by replacement of the media with StemFlex Media (Thermo Fisher Scientific #A3349401). Individual colonies of approximately 3,000-5,000 cells were then lifted and transferred to ultra low-adhesion plates (Millipore Sigma #CLS3471) in telencephalon differentiation media containing Glasgow Minimum Essential Medium (Thermo Fisher Scientific #11710035), 10% Knockout Serum Replacement (Thermo Fisher Scientific # 10828028), 0.1 mM MEM Non-Essential Amino Acids (Thermo Fisher Scientific #11140050), 1 mM Sodium Pyruvate (Millipore Sigma #S8636), 0.1 mM 2-Mercaptoethanol (Millipore Sigma #M3148) and 100 μg/ml Primocin (Invivogen #ant-pm-05). Telencephalon differentiation medium was supplemented with fresh Rho Kinase Inhibitor (Y-27632, 10 μM, Stem Cell Technologies #72304), WNT inhibitor (IWR1-8, 3 M, Cayman Chemical #13659) and TGF-Beta pathway inhibitor (SB431542, Tocris #1614, 5 μM). The plates were placed on an orbital shaker at 90 revolutions per minute. Organoids were grown under 20% O₂, 5% CO₂ and 37° C. conditions. Media was changed every other day from days 0-7.

On day 7, organoids were transferred to neuronal differentiation medium containing Dulbecco's Modified Eagle Medium: Nutrient Mixture F-12 with GlutaMAX supplement (Thermo Fisher Scientific #10565018), 1X N-2 Supplement (Thermo Fisher Scientific #17502048), 1X Chemically Defined Lipid Concentrate (Thermo Fisher Scientific #11905031) and 100 μg/ml Primocin. Medis was changed every 2-3 days.

On day 14 onward, 5 μg/mL Heparin sodium salt from porcine intestinal mucosa (Millipore Sigma #H3149), and 0.5% v/v Matrigel Growth Factor Reduced (GFR) Basement Membrane Matrix, LDEV-free (Matrigel GFR, Corning #354230), were added to the neuronal differentiation medium.

On day 32 onward, the organoids were transferred to neuronal maturation media containing BrainPhys Neuronal Medium (Stem Cell Technologies #05790), 1X N-2 Supplement, 1X Chemically Defined Lipid Concentrate (Thermo Fisher Scientific #11905031), 1X B-27 Supplement (Thermo Fisher Scientific #17504044), and 100 μg/ml Primocin and 1% v/v Matrigel Growth Factor Reduced (GFR) Basement Membrane Matrix, LDEV-free.

Brightfield images of telencephalic chicken organoids generated using passage 6 HE2M38 cells are provided in FIG. 5.

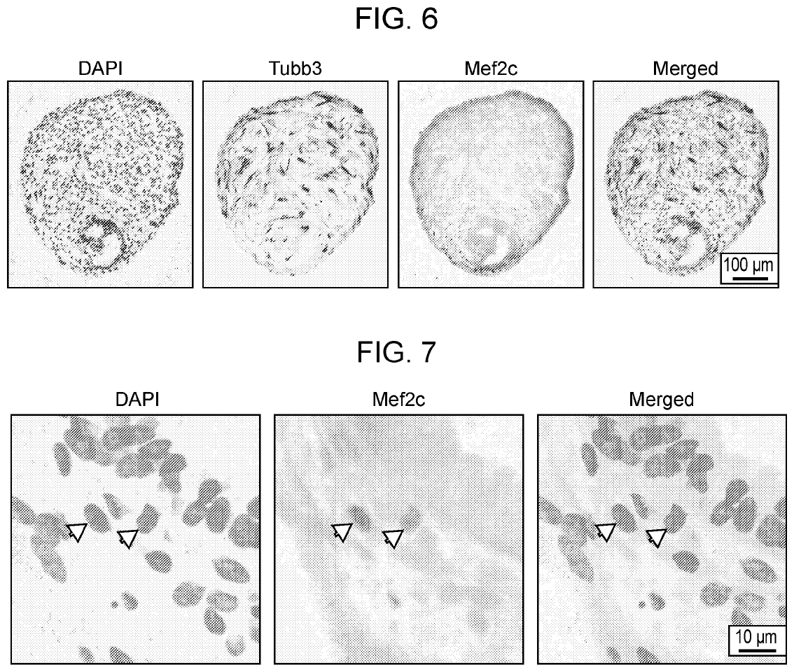

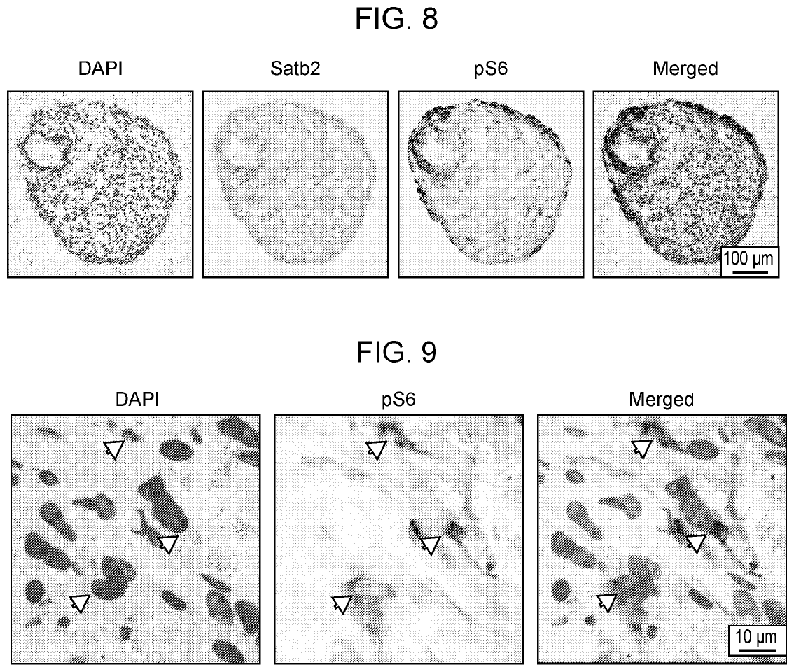

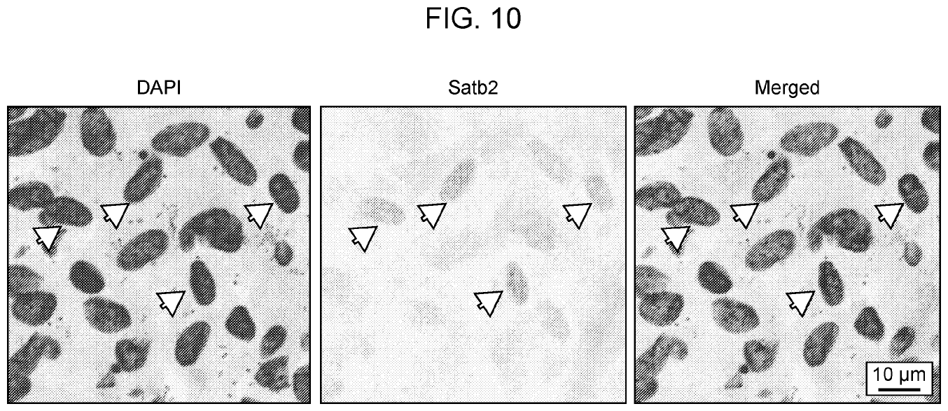

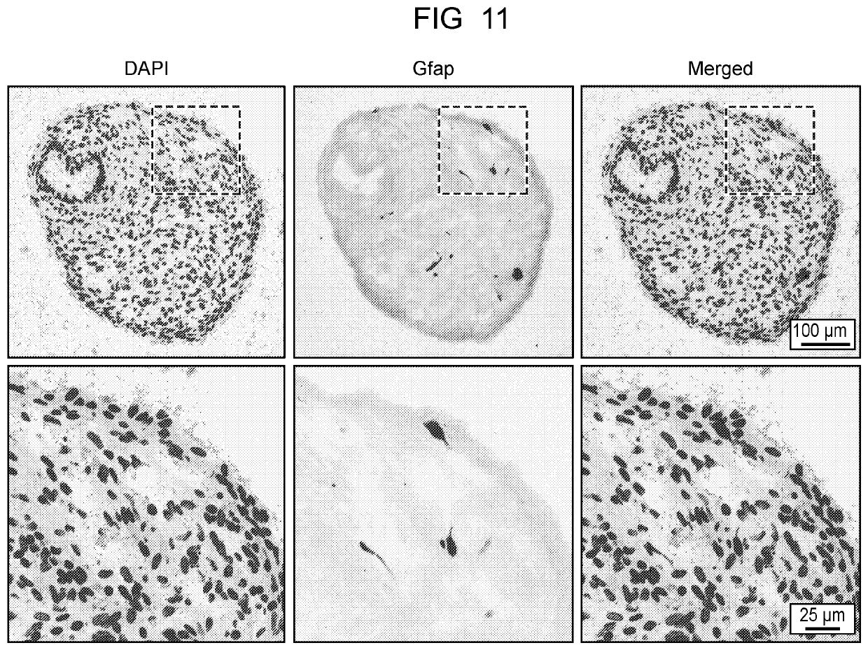

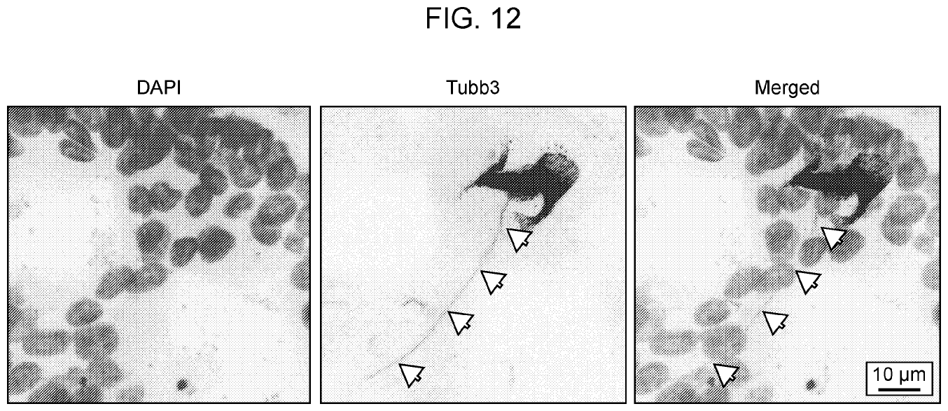

Immunohistochemical staining for various markers of interest was performed at differentiation day 17. FIG. 6 shows staining results for the neuronal markers Tubb3 and Mef2c at lower magnification. FIG. 7 shows staining results for Mef2c at higher magnification. FIG. 8 shows staining results for the transcription factor SATB2 and phosphorylated ribosomal protein S6 (pS6) at lower magnification. FIGS. 9 and 10 show staining results for pS6 and SATB2, respectively, at higher magnification. SATB2 labels a subset of excitatory neurons in the dorsal pallium of the telencephalon. FIGS. 11 and 12 show staining results for glial fibrillary acidic protein (GFAP) and neuronal-specific microtubule protein beta tubulin class III (Tubb3), respectively. GFAP labels astroglial cells in the brain, while Tubb3 labels axons.

Altogether, the presence of the above-mentioned immunohistochemistry markers confirms the regional identity and cell diversity of telencephalic organoids. Specifically, the organoids contain pS6, a downstream target of the mTOR pathway, which is a highly conserved in the dorsal pallium of the telencephalon (Pollen et al. (2019) Cell 176(4):743-756). They contain the presence of neurons, labeled by Tubb3 (Lee et al. (1990) Cell motility and the cytoskeleton 17(2):118-132), and astroglial cells marked with GFAP (Lee et al. (2008) Glia, 56(5):481-493). A subset of the neurons, express the neuronal maturation marker Mef2c (Pollen et al. (2019) Cell 176(4):743-756), and a subset of neurons express the marker Satb2, which specifically labels neurons of dorsal pallium in the avian telencephalon (Nomura et al. (2018) Cell reports, 22(12):3142-3151). It can therefore be concluded that the organoids generated through these methods contain a diverse set of cells of telencephalic fate, including maturing and mature neurons of the dorsal pallium, as well as astroglia cells.

### Methods

### Immunohistochemistry

Cells were washed and fixed in 4% Paraformaldehyde (PFA) (Thermo Fisher Scientific #28908) for 45 minutes at room temperature and washed 3 times in 1X Phosphate-buffered saline (PBS) pH 7.4 (Thermo Fisher Scientific #70011044). 10 minutes each time.

Organoids were collected and fixed in 4% (PFA) for 45 minutes at room temperature and cryopreserved in 30% Sucrose (Millipore Sigma #S8501). They were then embedded in a solution containing 50% of Tissue-Tek O.C.T. Compound (Sakura #4583) and 50% of 30% sucrose dissolved in 1X PBS pH 7.4. They were then sectioned to 12 μm using a cryostat (Leica Biosystems #CM3050) directly onto glass slides. After 3 washes of 10 minutes in 1X PBS, the sections were incubated in blocking solution 5% v/v donkey serum (Millipore Sigma #D9663), 2% gelatin w/v (Millipore Sigma #G9391) and 0.1% Triton X-100 (Millipore Sigma #X100) for 1 hour.

Cells and organoid sections were then incubated in primary antibodies overnight at 4° C. They were then washed 3 times for 30 minutes and incubated in secondary antibodies for 90 minutes at room temperature. They were then washed 3 times for 30 minutes in PBS and one time in sterile water for 10 minutes.

Primary antibodies used were: mouse anti GFAP (Millipore Sigma #G6171); rabbit anti MEF2C (Abcam #ab227085, 1:150); mouse anti SATB2 (Abcam #ab51502, 1:100); rabbit anti phosphorylated S6 ribosomal protein (Ser235/236) (Cell Signaling Technology #2211, 1:150); mouse anti TUBB3 (Biolegend #801201, 1:150). Secondary antibodies were of the Alexa series (Thermo Fisher Scientific), used at a concentration of 1:250. Nuclear counterstain was performed using 300 nM DAPI (4′, 6-Diamidino-2-Phenylindole, Dihydrochloride) (Thermo Fisher Scientific #D1306).

### Alkaline Phosphatase Staining

Alkaline phosphatase staining on live cells was performed using an alkaline phosphatase staining kit (Abcam #ab284936). First, cells were washed by incubating them in Wash Buffer for 2 minutes at room temperature. Then, the cells were incubated in freshly resuspended AP Staining Reagent at 37° C. for 30 minutes. Finally, the cells were washed using Wash buffer for 1 minute at room temperature.

Immediately after washing, the cells were fixed in 4% paraformaldehyde (Thermo Fisher Scientific #28908) for 45 minutes at room temperature. The samples were then washed in PBS 3 times for 10 minutes each time.

### Imaging

All brightfield imaging was performed using the EVOS M7000 Imaging system. Fluorescent confocal images were obtained using a Zeiss LSM 880 microscope with manual definition of the z-stacks. Image processing was done using FIJI (National Institutes of Health) and imported into Adobe Illustrator 2021.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of producing avian embryonic stem cells, the method comprising:
culturing epiblast cells obtained from a stage IX-XI avian embryo in a cell culture medium present in a feeder-free cell culture container.

2. The method according to claim 1, wherein the cell culture medium comprises active FGF-2.

3. The method according to claim 1, wherein the cell culture medium comprises a cell-permeable MEK/ERK pathway inhibitor.

4. The method according to claim 3, wherein the cell-permeable MEK/ERK pathway inhibitor is present in an amount of from 0.01 to 1 μM.

5. The method according to claim 1, wherein the cell culture medium comprises a TGF-beta type I receptor signaling inhibitor.

6. The method according to claim 5, wherein the TGF-beta type I receptor signaling inhibitor is present in an amount of from 0.1 to 10 μM.

7. The method according to claim 1, wherein the cell culture medium comprises a cell-permeable glycogen synthase kinase 3 (GSK-3) inhibitor.

8. The method according to claim 7, wherein the GSK-3 inhibitor is present in an amount of from 0.1 to 10 μM.

9. The method according to claim 1, wherein the cell culture medium comprises Leukemia Inhibitory Factor (LIF).

10. The method according to claim 9, wherein the LIF is chicken LIF.

11. The method according to claim 9 or 10, wherein the LIF is present in an amount of from 25 to 75 U/ml.

12. The method according to claim 1, wherein the cell culture medium comprises a cell-permeable Rho-associated, coiled-coil containing protein kinase (ROCK) inhibitor.

13. The method according to claim 12, wherein the cell-permeable ROCK inhibitor is present in an amount of from 5 to 15 μM.

14. The method according to claim 1, wherein the cell culture medium comprises chicken serum.

15. The method according to claim 14, wherein the chicken serum is present in an amount of from 5 to 15%.

16. The method according to claim 1, wherein the feeder-free cell culture container is a glycoprotein-coated cell culture container.

17. The method according to claim 16, wherein the feeder-free cell culture container is a vitronectin (VTN-N)-coated cell culture container.

18. The method according to claim 1, wherein the avian embryo is a chicken embryo.

19. A method comprising genetically modifying avian embryonic stem cells produced according to the methods of any one of claims 1 to 18.

20. Avian embryonic stem cells produced according to the method of any one of claims 1 to 18.

21. A method comprising differentiating the avian embryonic stem cells of claim 20 into cells of an organ.

22. Cells of an organ produced according to the method of claim 21.

23. A method comprising producing a two-dimension culture, a spheroid, or an organoid from the avian embryonic stem cells of claim 20.

24. The method according to claim 23, wherein the method comprises producing an organoid from the avian embryonic stem cells.

25. The method according to claim 24, wherein the organoid is an avian telencephalic organoid.

26. The method according to claim 25, wherein the method comprises culturing the avian embryonic stem cells in a telencephalon differentiation medium comprising a ROCK inhibitor, a WNT inhibitor, a TGF-β pathway inhibitor, or any combination thereof.

27. A spheroid or organoid produced according to the method of any one of claims 23 to 26.

28. A method comprising engrafting into a non-human animal the avian embryonic stem cells of claim 20.

29. A method comprising engrafting into a non-human animal the cells of an organ of claim 22.

30. A method comprising engrafting into a non-human animal the spheroid or organoid of claim 27.

31. A method comprising engrafting into a fertilized egg or an unfertilized egg the avian embryonic stem cells of claim 20.

32. A screening method comprising:
contacting avian embryonic stem cells produced according to the method of any one of claims 1 to 18 with a test agent; and
assessing for an effect of the test agent on the avian embryonic stem cells.

33. A screening method comprising:
contacting spheroids or organoids produced according to the method of any one of claims 23 to 26 with a test agent; and
assessing for an effect of the test agent on the spheroids or organoids.

34. The method according to claim 33, wherein an avian telencephalic organoid produced according to the method of any one of claims 23 to 26 is contacted with the test agent.


SEQUENCE LISTING (3 sequences)

SEQ ID NO: 1 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 2 - protein, 203 aa, Mus musculus [sequence omitted]

SEQ ID NO: 3 - protein, 211 aa, Gallus gallus
MRLIPAGVVPFVALLLLQRRPVSGRALLGTSSACPTNGLCRANVLEQTRRQVALLNATAQDLFSLYLKCQGEPFSSESDRLCSPSGIFFPPFHVNRTTERKEVMVAMYKLFAFLNASLGNITRDQEELNPMAKELLNRLHNTTKTTRGLISNLTCLLCKHYNIFQVDVSYGESSKDKSAFKKKQQGCQVLRKYVQVIAQAARVLLPHLSPA